(19) United States
(12) Patent Application Publication
(10) Pub. No.: US 2024/0389904 A1
(43) Pub. Date: Nov. 28, 2024

(54) IMPLANTABLE CHEMICAL SENSOR PACKAGES WITH CALIBRATION FEATURES

(71) Applicant: Cardiac Pacemakers, Inc., St. Paul, MN (US)

(72) Inventors: Yingbo Li, Woodbury, MN (US); Michael J. Kane, St. Paul, MN (US)

(21) Appl. No.: 18/669,801

(22) Filed: May 21, 2024

Related U.S. Application Data
(60) Provisional application No. 63/468,143, filed on May 22, 2023.

(51) Int. Cl. A61B 5/1473; A61B 5/00; A61B 5/1495

(52) U.S. Cl. CPC A61B 5/1473; A61B 5/1495; A61B 5/6861; A61B 2560/0252

(57) ABSTRACT

Embodiments herein relate to embodiments herein relate to packages for implantable chemical sensors with features to facilitate calibration of the sensors therein. In an embodiment, a chemical sensor system is included having a package and an implantable monitor device therein. The implantable monitor device can include an optical chemical sensor. A first aqueous solution including a solute can be disposed within the package. In some embodiments, the package includes a first chamber a second chamber separated by a frangible seal. A first aqueous solution can be disposed within the first chamber and a second aqueous solution can be disposed in the second chamber. Other embodiments are also included herein.

This application claims the benefit of U.S. Provisional Application No. 63/468,143, filed May 22, 2023, the content of which is herein incorporated by reference in its entirety.

### FIELD

Embodiments herein relate to packages for implantable chemical sensors. More particularly, embodiments herein relate to packages for implantable chemical sensors with features to facilitate calibration of the sensors therein.

### BACKGROUND

Data regarding physiological analytes are highly relevant for the diagnosis and treatment of many conditions and disease states. As one example, potassium ion concentrations can affect a patient's cardiac rhythm. Therefore, medical professionals frequently evaluate physiological potassium ion concentration when diagnosing a cardiac rhythm problem. However, measuring physiological concentrations of analytes, such as potassium, generally requires drawing blood from the patient. Blood draws are commonly done at a medical clinic or hospital and therefore generally require the patient to physically visit a medical facility. As a result, despite their significance, physiological analyte concentrations are frequently measured only sporadically.

### SUMMARY

Embodiments herein relate to packages for implantable chemical sensors with features to facilitate calibration of the sensors therein. In a first aspect, a chemical sensor system can be included having a package and an implantable monitor device. The implantable monitor device can include an optical chemical sensor. The implantable monitor device can be disposed within the package. A first aqueous solution can be disposed within the package. The first aqueous solution can include a solute.

In a second aspect, in addition to one or more of the preceding or following aspects, or in the alternative to some aspects, the package can include at least one selected from the group consisting of a polymeric film, a polymeric film bag, and a polymeric tube.

In a third aspect, in addition to one or more of the preceding or following aspects, or in the alternative to some aspects, the package can be hermetically sealed.

In a fourth aspect, in addition to one or more of the preceding or following aspects, or in the alternative to some aspects, the solute can include an electrolyte.

In a fifth aspect, in addition to one or more of the preceding or following aspects, or in the alternative to some aspects, the electrolyte can include potassium.

In a sixth aspect, in addition to one or more of the preceding or following aspects, or in the alternative to some aspects, the first aqueous solution can have a pH of 7.0 to 7.8.

In a seventh aspect, in addition to one or more of the preceding or following aspects, or in the alternative to some aspects, the first aqueous solution can be pH buffered.

In an eighth aspect, in addition to one or more of the preceding or following aspects, or in the alternative to some aspects, the optical chemical sensor can include a hydrogel layer.

In a ninth aspect, in addition to one or more of the preceding or following aspects, or in the alternative to some aspects, the package can include a first chamber and a second chamber. The package can include a seal. The first aqueous solution can be disposed within the first chamber. The chemical sensor system can further include a second aqueous solution, wherein the second aqueous solution can be disposed in the second chamber. The seal can separate the first chamber and the second chamber and can be frangible.

In a tenth aspect, in addition to one or more of the preceding or following aspects, or in the alternative to some aspects, the system can further include a temperature sensor, wherein the temperature sensor can be disposed on or in the package.

In an eleventh aspect, a chemical sensor system can be included having a package with a first chamber, a second chamber, and a seal. The seal can separate the first chamber and the second chamber. The seal can be frangible. An implantable monitor device can be within the first chamber of the package. The implantable monitor device can include an optical chemical sensor. A first aqueous solution can be disposed within the second chamber. The first aqueous solution can include a solute.

In a twelfth aspect, in addition to one or more of the preceding or following aspects, or in the alternative to some aspects, the package can further include at least one selected from the group consisting of a polymeric film, a polymeric film bag, and a polymeric tube.

In a thirteenth aspect, in addition to one or more of the preceding or following aspects, or in the alternative to some aspects, the package can be hermetically sealed.

In a fourteenth aspect, in addition to one or more of the preceding or following aspects, or in the alternative to some aspects, the solute can include an electrolyte.

In a fifteenth aspect, in addition to one or more of the preceding or following aspects, or in the alternative to some aspects, the electrolyte can include potassium.

In a sixteenth aspect, in addition to one or more of the preceding or following aspects, or in the alternative to some aspects, the first aqueous solution can have a pH of 7.0 to 7.8.

In a seventeenth aspect, in addition to one or more of the preceding or following aspects, or in the alternative to some aspects, the first aqueous solution can be pH buffered.

In an eighteenth aspect, in addition to one or more of the preceding or following aspects, or in the alternative to some aspects, the first aqueous solution can have a first color.

In a nineteenth aspect, in addition to one or more of the preceding or following aspects, or in the alternative to some aspects, the optical chemical sensor can include a hydrogel layer.

In a twentieth aspect, in addition to one or more of the preceding or following aspects, or in the alternative to some aspects, the package the chemical sensor system can further include a third chamber, and a second seal, wherein the second seal can be frangible. The chemical sensor system can further include a second aqueous solution, wherein the second aqueous solution can be disposed within the third chamber. The second aqueous solution can include a solute, wherein the solute of the second aqueous solution can be at a different concentration than in the first aqueous solution.

In a twenty-first aspect, in addition to one or more of the preceding or following aspects, or in the alternative to some aspects, the second aqueous solution can have a second color.

In a twenty-second aspect, in addition to one or more of the preceding or following aspects, or in the alternative to some aspects, can further include a temperature sensor, wherein the temperature sensor can be disposed on or in the package.

In a twenty-third aspect, in addition to one or more of the preceding or following aspects, or in the alternative to some aspects, the temperature sensor can be passive.

In a twenty-fourth aspect, in addition to one or more of the preceding or following aspects, or in the alternative to some aspects, the temperature sensor can be disposed over or in at least part of each of the chambers.

In a twenty-fifth aspect, a method of calibrating an implantable chemical sensor can be included. The method can include breaking a first seal of the package to provide fluid communication between the first chamber and a second chamber of the package, measuring a concentration of the solute in the first chamber of the package, and comparing the measured concentration in the first chamber against a known concentration of the solute.

In a twenty-sixth aspect, in addition to one or more of the preceding or following aspects, or in the alternative to some aspects, the method can further include measuring a concentration of a solute in a first chamber of a package prior to the breaking a first seal of the package to provide fluid communication between the first chamber and a second chamber of the package.

In a twenty-seventh aspect, in addition to one or more of the preceding or following aspects, or in the alternative to some aspects, the method can further include determining a calibration correction based on the compared concentration.

In a twenty-eighth aspect, in addition to one or more of the preceding or following aspects, or in the alternative to some aspects, the method can further include waiting for the implantable chemical sensor to reach an equilibrium value after the operation of breaking the first seal of the package and determining a response time of the implantable chemical sensor based on an elapsed time.

In a twenty-ninth aspect, in addition to one or more of the preceding or following aspects, or in the alternative to some aspects, can further include breaking a second seal of the package to provide fluid communication between the first chamber and a third chamber of the package and measuring a concentration of the solute in the first chamber of the package.

In a thirtieth aspect, in addition to one or more of the preceding or following aspects, or in the alternative to some aspects, the method can further include comparing the measured concentration in the first chamber against a known concentration of the solute.

In a thirty-first aspect, in addition to one or more of the preceding or following aspects, or in the alternative to some aspects, the method can further include waiting for the implantable chemical sensor to reach an equilibrium value after the operation of breaking the second seal of the package and determining a response time of the implantable chemical sensor based on an elapsed time.

In a thirty-second aspect, in addition to one or more of the preceding or following aspects, or in the alternative to some aspects, the first chamber can be filled with a liquid prior to the breaking a first seal of the package to provide fluid communication between the first chamber and a second chamber of the package.

In a thirty-third aspect, in addition to one or more of the preceding or following aspects, or in the alternative to some aspects, the first chamber is not filled with a liquid prior to the breaking a first seal of the package to provide fluid communication between the first chamber and a second chamber of the package.

In a thirty-fourth aspect, in addition to one or more of the preceding or following aspects, or in the alternative to some aspects, the method can further include measuring temperature and adjusting a response time and/or a response time comparison value based on the same.

In a thirty-fifth aspect, in addition to one or more of the preceding or following aspects, or in the alternative to some aspects, the method can further include measuring and/or receiving a temperature value and terminating calibration if the temperature value can be not within a predetermined range.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope herein is defined by the appended claims and their legal equivalents.

### BRIEF DESCRIPTION OF THE FIGURES

Aspects may be more completely understood in connection with the following figures (FIGS.), in which:

FIG. 1 is a schematic view of an implantable monitor device implanted in a patient in accordance with various embodiments herein.

FIG. 2 is a schematic view of an implantable monitor device in accordance with various embodiments herein.

FIG. 3 is a schematic view of a package for an implantable monitor device in accordance with various embodiments herein.

FIG. 4 is a schematic view of an implantable sensor system including an implantable monitor device within a package in accordance with various embodiments herein.

FIG. 5 is a schematic view of an implantable sensor system including an implantable monitor device within a package in accordance with various embodiments herein.

FIG. 6 is a schematic view of an implantable sensor system including an implantable monitor device within a package in accordance with various embodiments herein.

FIG. 7 is a schematic view of an implantable sensor system including an implantable monitor device within a package in accordance with various embodiments herein.

FIG. 8 is a sensor response curve in accordance with various embodiments herein.

FIG. 9 is a sensor response curve in accordance with various embodiments herein.

FIG. 10 is a sensor response curve in accordance with various embodiments herein.

FIG. 11 is a schematic view of an implantable sensor system including an implantable monitor device within a package in accordance with various embodiments herein.

FIG. 12 is a cross-sectional schematic view of an implantable monitor device in accordance with various embodiments herein.

FIG. 13 is a block diagram of an implantable monitor device in accordance with various embodiments herein.

FIG. 14 is a schematic view of an implantable sensor system including an implantable monitor device within a package in accordance with various embodiments herein.

FIG. 15 is a schematic view of an implantable sensor system including an implantable monitor device within a package in accordance with various embodiments herein.

FIG. 16 is a schematic view of an implantable sensor system including an implantable monitor device within a package in accordance with various embodiments herein.

While embodiments are susceptible to various modifications and alternative forms, specifics thereof have been shown by way of example and drawings, and will be described in detail. It should be understood, however, that the scope herein is not limited to the particular aspects described. On the contrary, the intention is to cover modifications, equivalents, and alternatives falling within the spirit and scope herein.

### DETAILED DESCRIPTION

Data regarding physiological analytes are highly relevant for the diagnosis, treatment, and/or monitoring of many conditions and disease states. However, in vitro assays to determine physiological analyte values have substantial limitations. In contrast, implantable chemical sensors can be used to gather data about physiological analytes while a patient is away from a medical care facility and without needing to draw blood or another fluid from the patient. However, implantable chemical sensors have their own challenges. For example, generally there is a need to calibrate implantable chemical sensors prior to or at the time of implant.

Embodiments herein relate to implantable chemical sensor packages with features to aid in the calibration process and implantable chemical sensor systems including the same. Implantable monitor devices with chemical sensors can be stored within a package after manufacturing until the time of implant into a patient. The packages can include aqueous solutions therein. In some embodiments an aqueous solution can bathe the implantable monitor device as it sits in the package during storage (“wet shipped”). In other embodiments, a chamber within the package that holds the implantable monitor device during storage is dry (“dry shipped”). Either way, the packages can include one or more frangible seals that connect to additional chambers that are separate from the main chamber where the device sits during storage.

The seals can be broken or otherwise opened as a part of preparations for implanting the monitor device. Breaking or otherwise opening the seals can bring the separate chambers into fluid communication with the main chamber and allow for a separate aqueous solution to mix with a solution/liquid in the main chamber (in the case of “wet shipped”) or allow an aqueous solution to flow into the main chamber and wet a chemical sensor of the monitor device (in the case of “dry shipped”). This will cause (in either case) the optical chemical sensor to be exposed to a known solute concentration. Measurements can be taken using the implantable monitor device and then compared with the known concentration of solute. If needed, calibration adjustments (such as offset values or the like) can then be made. In addition, measurements of sensor response time can be made. In some embodiments, measured sensor response times can be measured against predetermined values to determine if the response time is satisfactory.

In some embodiments, the package may contain one or more chambers that are initially sealed off with a frangible seal. Different solutions having different solute concentrations can be stored in the different chambers that are initially sealed off. In this scenario, a multi-step calibration procedure can be performed. For example, a first frangible seal can be broken or opened causing the sensor to be exposed to a solution with a first solute concentration. Then, after allowing time for the sensor to respond to that change, a second frangible seal can be broken or opened causing the sensor to be exposed to a solution with a second solute concentration. Further details are provided as follows.

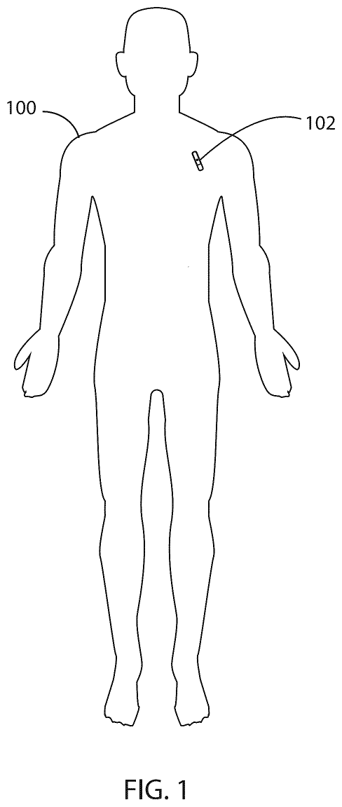

Referring now to FIG. 1, a schematic view of an implantable monitor device 102 implanted in a patient 100 is shown in accordance with various embodiments herein. The implantable monitor device 102 can be part of an implantable sensor system herein. The implantable monitor device 102 can be configured to sense concentrations of various analytes of interest. In some embodiments the chemical sensor element can be configured to detect one or more of an electrolyte, a protein, a sugar, a hormone, a peptide, an amino acid and a metabolic product. In some embodiments, the chemical sensor element can be configured to detect an ion selected from a group consisting of potassium, sodium, chloride, calcium, magnesium, lithium, hydronium, hydrogen phosphate, bicarbonate, and the like. In some embodiments, the chemical sensor element can be configured to detect any of the previous ions other than hydronium or hydrogen ion. It will be appreciated that many other chemical analytes are also contemplated herein.

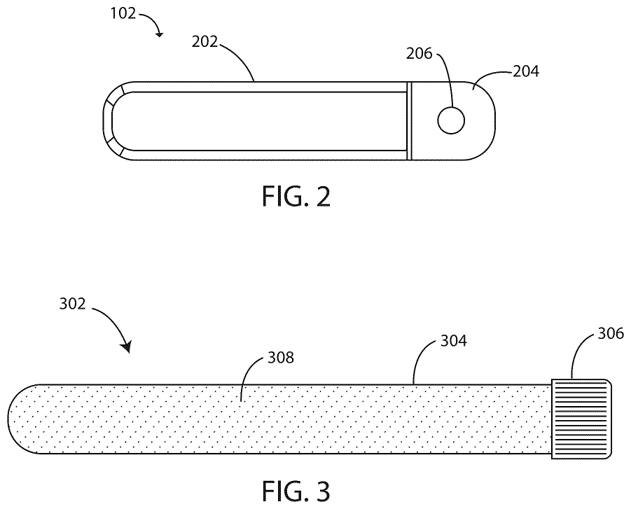

Referring now to FIG. 2, an implantable monitor device 102 is shown in accordance with various embodiments herein. The implantable monitor device 102 can include an implantable housing 202 and a header 204 coupled to the implantable housing 202. Various materials can be used for the implantable housing 202. However, in some embodiments, the implantable housing 202 can be formed of a material such as a metal, ceramic, a polymer, or a composite. The header 204 can be formed of various materials, but in some embodiments the header 204 can be formed of a polymer (translucent or opaque) such as an epoxy material. In some embodiments the header 204 can be hollow. In other embodiments the header 204 can be filled with components and/or structural materials such as epoxy or another material such that it is non-hollow. In some embodiments, however, a distinct header 204 can be omitted. Rather, the implantable housing 202 can include substantially all of the components of the device.

The implantable monitor device 102 can also include an optical chemical sensor 206 coupled to the implantable housing 202. The chemical sensor element(s) can be configured to detect an analyte concentration within a bodily fluid when implanted in the body. Bodily fluids can include blood, interstitial fluid, serum, lymph, serous fluid, cerebrospinal fluid, and the like.

It will be appreciated that the optical chemical sensor 206 can be positioned at any location along implantable monitor device 102, including along the implantable housing 202 or along the header 204. It will also be appreciated that although FIG. 2 shows a device having one sensor 206, any number of sensors can be present. For example, the device can include at least 1, 2, 3, 4, 5, 6, 7, 8, 9, 10, 11, 12, or more sensors, or a number of sensors falling within a range between any of the foregoing.

The implantable monitor device 102 can take on various dimensions. In some embodiments herein it can be approximately 2 to 3 inches in length, 0.4 to 0.6 inches wide, and 0.15 to 0.35 inches thick. However, in some embodiments, the implantable monitor device 102 can be about 0.25, 0.50, 1.0, 2.0, 3.0, 4.0, or 5.0 inches in length. In some embodiments the length can be in a range wherein any of the foregoing lengths can serve as the upper or lower bound of the range, provided that the upper bound is greater than the lower bound. In some embodiments, the implantable monitor device 102 can be about 0.25, 0.50, 0.75, 1.0, or 2.0 inches in width. In some embodiments the length can be in a range wherein any of the foregoing lengths can serve as the upper or lower bound of the range, provided that the upper bound is greater than the lower bound. In some embodiments, the implantable monitor device 102 can be about 0.10, 0.25, 0.50, 0.75, or 1.0 inches thick. In some embodiments the thickness can be in range wherein any of the foregoing thickness can serve as the upper or lower bound of the range, provided that the upper bound is greater than the lower bound.

Referring now to FIG. 3, a schematic view of a package 302 for an implantable monitor device 102 is shown in accordance with various embodiments herein. In this example, the package 302 includes a body 304 and a removeable cap 306 and takes the form of a polymeric tube. However, many different package forms are contemplated herein. In some embodiments, the package can be formed with a polymeric film. In various embodiments, the package can include a polymeric film bag (such as a MYLAR bag), a covered tray, or the like. In various embodiments, the package can be hermetically sealed to maintain sterility until the time for surgical implantation.

In some embodiments, the package 302 includes an aqueous solution 308 disposed therein. In various embodiments, the aqueous solution can include a solute therein. The solute can correspond to the analyte that the chemical sensor is configured to detect. For example, if the chemical sensor is configured to detect potassium, then the solute within the aqueous solution 308 can be potassium. The concentration of the solute within the aqueous solution can be selected and set to a specific value when the solution is prepared. In some embodiments, the concentration of the solute can be at or near physiological levels, but in some embodiments the concentration can be higher or lower. For example, in embodiments where the solute is potassium, the concentration can be from 3.5 to 5.0 mmol/liter. However, in some embodiments, the solute concentration (wherein the solute is any of those referred to herein) can be higher than physiological levels and in some embodiments the concentration can be lower than physiological levels. For example, where the solute is potassium the concentration can be greater than 5.0 mmol/liter in some embodiments and in other embodiments the concentration can be less than 3.5 mmol/liter. In embodiments where more than one solution is used, the concentrations of solute can be the same or different between the solutions used. For example, in some embodiments, the concentration of one solution used can be at a certain value and the concentration of another solution used can be higher or lower than the first solution.

In some embodiments, the aqueous solution has a pH from 7.0 to 7.8, but in other embodiments the aqueous solution can be more acidic or more basic. In some embodiments, the aqueous solution can be pH buffered. However, in some embodiments, a portion of the package 302 initially holding the implantable monitor device 102 is not filled with a liquid.

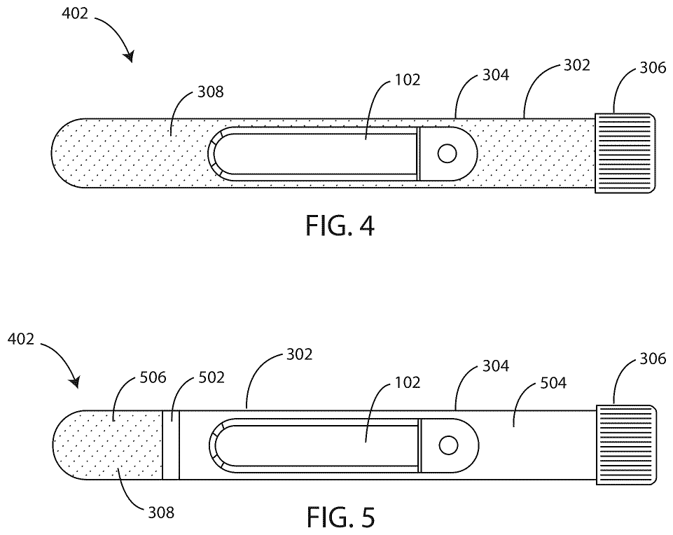

The implantable monitor device 102 can be disposed inside of the package 302. Referring now to FIG. 4, a schematic view of an implantable sensor system 402 including implantable monitor device 102 within a package 302 is shown in accordance with various embodiments herein. As before, the package 302 includes a body 304, a cap 306, and an aqueous solution 308 disposed therein. In this embodiment, the chemical sensor of the implantable monitor device 102 is bathed within the aqueous solution 308. The presence of the aqueous solution 308 with a solute dissolved therein allows for a single point of reference in a calibration procedure. For example, the implantable monitor device 102 can be turned on and a measured concentration of solute can be compared with the known concentration of solute in the aqueous solution 308 and adjustments, if any are needed, can be preformed as part of a calibration operation.

In some embodiments, a package 302 herein can include a frangible seal. This can allow for multi-point calibration procedures (versus a single point of reference). This can also allow for dynamic response verification as the sensor changes from measuring a first concentration of solute to measuring a second concentration of solute. Referring now to FIG. 5, a schematic view of an implantable sensor system 402 including an implantable monitor device 102 within a package 302 is shown in accordance with various embodiments herein. As before, the package 302 includes a body 304, a cap 306, and an aqueous solution 308 disposed therein. However, in the embodiment of FIG. 5, the package 302 also includes a seal 502. The seal 502 divides a first chamber 504 from a second chamber 506. In various embodiments, the seal 502 can be frangible. In some embodiments, the seal 502 can include a membrane that is configured to rupture when a force is applied. In some embodiments, the seal 502 can include a brittle material that breaks when a force is applied. In some embodiments, the seal 502 can be formed by a relatively weak bond between sides of a package. In this example, when the seal 502 is broken, the aqueous solution 308 can flow into the first chamber 504 and bathe the chemical sensor of the implantable monitor device. The relative volume of the first chamber 504 can be adjusted to accommodate a desirable amount of the aqueous solution 308 (e.g., 1 ml, 5 ml, 10 ml, 25 ml, 50 ml, 100 ml, etc.). In some embodiments, first chamber 504 can be empty initially (such as with a “dry shipped” embodiment). However, in other embodiments, first chamber 504 can include pure water or an aqueous solution with a solute therein.

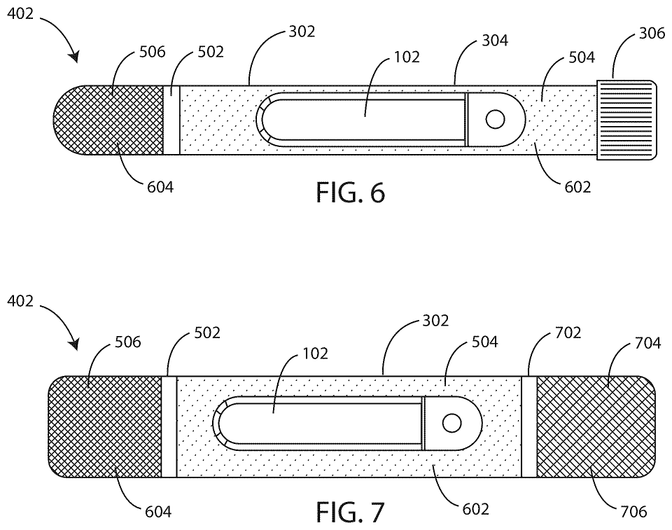

Referring now to FIG. 6, a schematic view of an implantable sensor system 402 including an implantable monitor device 102 within a package 302 is shown in accordance with various embodiments herein. As before, the package 302 includes a body 304, a cap 306, and an aqueous solution 308 disposed therein. The package 302 also includes a seal 502 that divides a first chamber 504 from a second chamber 506. The implantable monitor device 102 also includes a first aqueous solution 602 disposed in the first chamber 504. In some embodiments, the first aqueous solution 602 can have some concentration of solute. The implantable monitor device 102 also includes a second aqueous solution 604 disposed in the second chamber 506. The second aqueous solution 604 can have a different solute concentration than the first aqueous solution 602. In some embodiments, the second aqueous solution 604 can have a greater concentration of the solute than the first aqueous solution 602. In some embodiments, the second aqueous solution 604 can have a lesser concentration of the solute than the first aqueous solution 602, for example the second aqueous solution could be pure water. Regardless, when the seal 502 is broken, the concentration of the solution bathing the chemical sensor of the implantable monitor device 102 changes such that the accuracy of the sensor to changing concentrations and the response time can be measured.

In some embodiments herein, one or more chambers of the package (such as any of the chambers in any of the examples herein) can include a dry salt (such as a potassium salt, a sodium salt, a calcium salt, or the like) sealed in a compartment that is then dissolved by an aqueous fluid after a frangible seal is broken. For example, in the example of FIG. 6, the second chamber 506 may not initially include an aqueous solution, but rather contain a dry salt composition. The first chamber 504 can include an aqueous fluid, such as pure water, or water with a first salt content. Then when the seal 502 is broken, the aqueous fluid can dissolve the salt and the resulting solution can have a particular dissolved salt concentration and a particular pH. This resulting solution can bathe the implantable monitor device 102 therein.

In various embodiments, more than one frangible seal can be included allowing multi-stage calibration procedures to be performed. Referring now to FIG. 7, a schematic view of an implantable monitor device 102 within a package 302 is shown in accordance with various embodiments herein. The package 302 includes a seal 502 and a second seal 702. The package 302 includes a first chamber 504, a second chamber 506, and a third chamber 704. The implantable monitor device 102 also includes a first aqueous solution 602 disposed in the first chamber 504. The implantable monitor device 102 also includes a second aqueous solution 604 disposed in the second chamber 506. The implantable monitor device 102 also includes a third aqueous solution 706 disposed in the third chamber 704.

The first aqueous solution 602 can include a solute at a first concentration. The second aqueous solution 604 can include a solute at a second concentration. The third aqueous solution 706 can include a solute at a third concentration.

In various embodiments, the first seal 502 and the second seal 702 can be frangible. The first seal 502 and the second seal 702 can be configured such that they are separately openable. Thus, in use, the first seal 502 or second seal 702 can be opened first and then various operations can be performed while the other seal remains closed. Then, the other seal can be opened, and further operations can be performed.

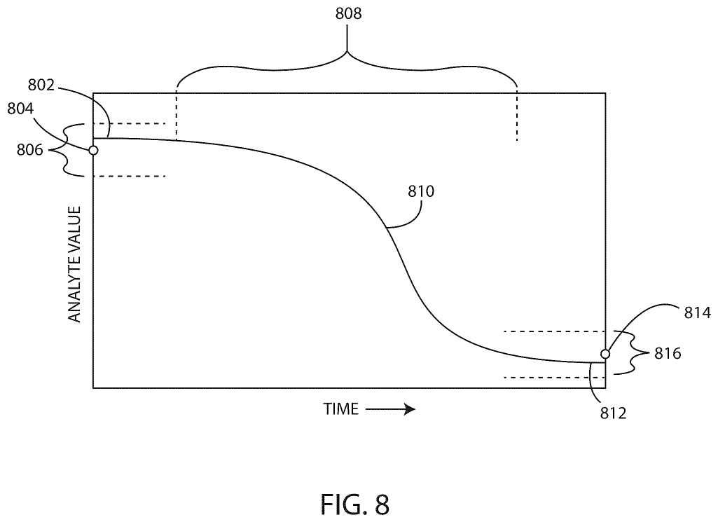

Referring now to FIG. 8, a sensor response curve is shown in accordance with various embodiments herein. FIG. 8 shows a measured starting solute concentration value 802 (such as measured with a chemical sensor system herein) along with an actual starting solute concentration value 804 (known based on the amount of solute in a solution herein). FIG. 8 also shows an allowed starting range 806. The allowed starting range 806 defines an acceptable level of measurement error. If the measured starting solute concentration value 802 falls within the starting range 806 then adjustments may not be necessary with respect to concentration values near the actual starting solute concentration value 804.

An action can be taken such that the chemical sensor is exposed to a solution with a different concentration of solute than it was initially exposed to. For example, a frangible seal can be broken or otherwise opened allowing the solute concentration around the chemical sensor to change. This will cause the sensor signal to change reflecting a changed concentration of solute. As such, FIG. 8 shows a measured ending value 812, an actual ending value 814, and an allowed ending range 816. If the measured ending solute concentration value 812 falls within the allowed ending range 816 then adjustments may not be necessary with respect to concentration values near the actual ending solute concentration value 814. However, if necessary, adjustments can be applied.

It will be appreciated that the change in measured concentration values is not instantaneous. As such, FIG. 8 shows a transition response 810. By monitoring the amount of time that elapses from an initial equilibrium value to a changed equilibrium value the system can derive a measured response time 808. The measured response time 808 can then be compared with a predetermined threshold value for response time to determine if the actual response time is satisfactory.

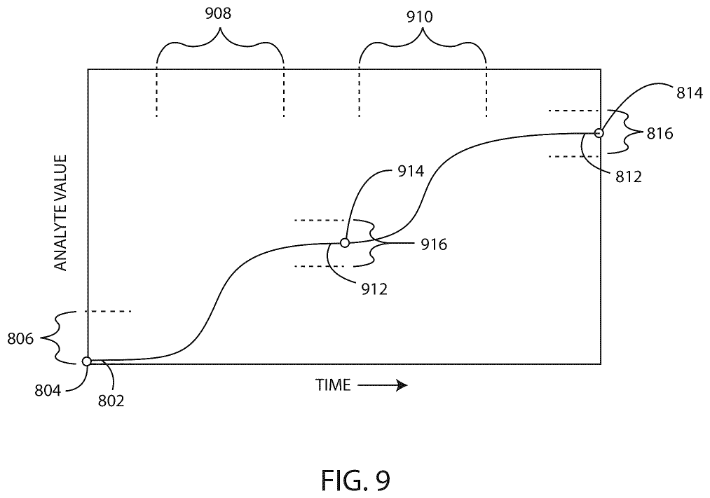

In some cases, a calibration procedure can include checking sensor-measured concentrations at more than one concentration value. Referring now to FIG. 9, a sensor response curve is shown in accordance with various embodiments herein. As with FIG. 8,

FIG. 9 shows a measured starting value 802 (such as measured with a chemical sensor system herein), an actual starting value 804, and an allowed starting range 806. Further, FIG. 9 shows a measured ending value 812, an actual ending value 814, and an allowed ending range 816.

However, in FIG. 9, a two-step change is illustrated. For example, a first frangible seal can be opened or broken in a first step and a second frangible seal can be opened or broken in a second step. As such, the concentration of the solute as exposed to the chemical sensor can change twice. Thus, FIG. 9 also shows an intermediate stage measured value 912, an intermediate stage actual value 914, and an intermediate stage allowed range 916. FIG. 9 also shows a first response time 908 and a second response time 910.

It will be appreciated that changes in solute concentration during calibration procedures herein can be both up and down. Thus, the solute concentration that the sensor is exposed to can be increased as well as decreased.

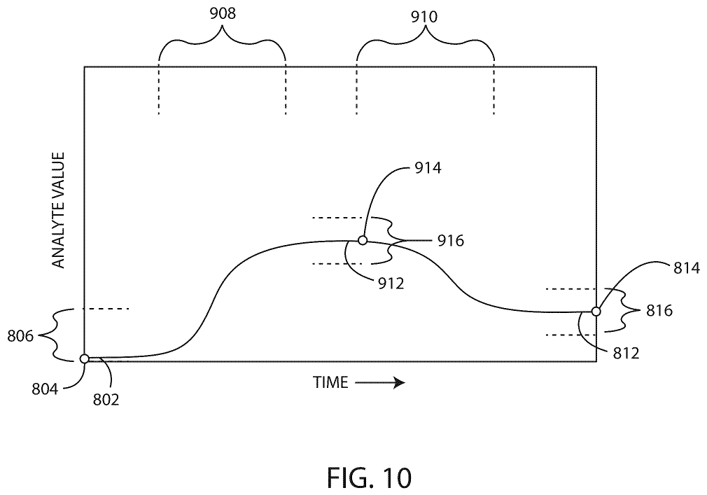

Referring now to FIG. 10, a sensor response curve is shown in accordance with various embodiments herein. As with FIG. 9, FIG. 10 shows a measured starting value 802 (such as measured with a chemical sensor system herein), an actual starting value 804, and an allowed starting range 806. Further, FIG. 10 shows a measured ending value 812, an actual ending value 814, and an allowed ending range 816. FIG. 10 also shows an intermediate stage measured value 912, an intermediate stage actual value 914, and an intermediate stage allowed range 916. FIG. 10 also shows a first response time 908 and a second response time 910. However, in this example, it will be appreciated that measured ending value 812 and actual ending value 814 are less than intermediate stage measured value 912 and intermediate stage actual value 914.

In some embodiments, the aqueous solutions can have different colors so that they can easily be distinguished from one another. For example, a dye (such as a biocompatible dye) can be added to one or more of the solutions. The colors can be selected so as not to interfere with optical sensors herein. Having different colors for the solutions can allow a user to visually distinguish one solution from another as well as have visual confirmation of solution mixing, such as after a frangible seal is broken open.

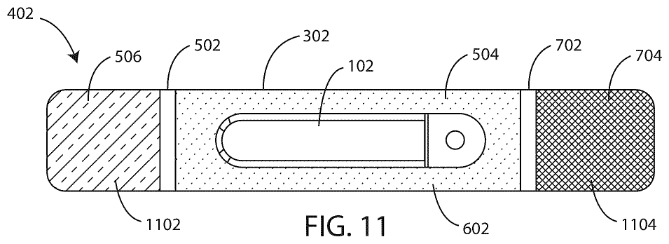

Referring now to FIG. 11, a schematic view of an implantable sensor system 402 including an implantable monitor device 102 within a package 302 is shown in accordance with various embodiments herein. As before, the package 302 includes a seal 502 and a second seal 702. The package 302 includes a first chamber 504, a second chamber 506, and a third chamber 704. The implantable monitor device 102 also includes an aqueous solution 602 disposed in the first chamber 504.

The implantable sensor system 402 also includes a first colored solution 1102 disposed in the second chamber 506. The implantable sensor system 402 also includes a second colored solution 1104 disposed in the third chamber 704. In some embodiments, the aqueous solution 602 may be colorless or have a color different than the first colored solution 1102 and the second colored solution 1104. When the seal 502 is broken, the first colored solution 1102 can mix with the aqueous solution 602 causing a visible color change. Similarly, when the second seal 702 is broken, the second colored solution 1104 can mix with the aqueous solution 602 causing another visible color change.

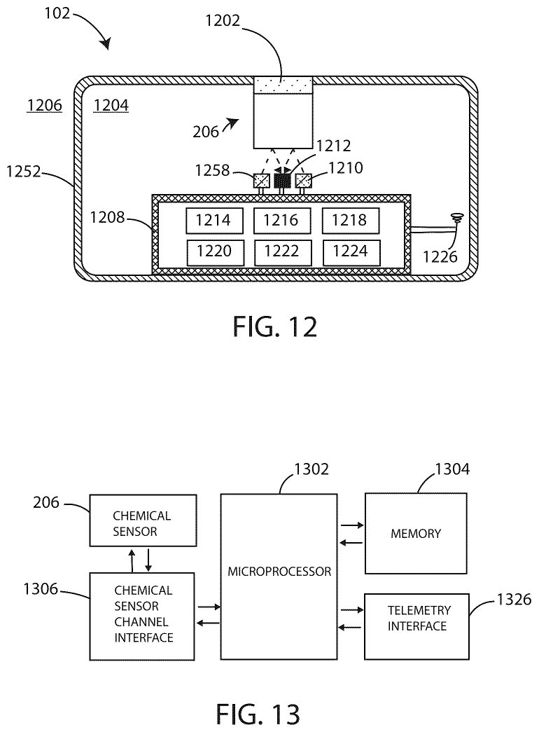

It will be appreciated that implantable medical devices herein can include many different components. Referring now to FIG. 12, a schematic view of implantable monitor device 102 is shown in accordance with various embodiments herein. The implantable monitor device 102 can include implantable housing 1252. The implantable housing 1252 of the implantable monitor device 102 can include various materials such as metals, polymers, ceramics, and the like. In some embodiments, the implantable housing 1252 can be a single integrated unit. In other embodiments, the implantable housing 1252 can include implantable housing 1252 and epoxy header 104, as discussed above. In some embodiments, the implantable housing 1252, or one or more portions thereof, can be formed of titanium. In some embodiments, one or more segments of the implantable housing 1252 can be hermetically sealed.

The implantable housing 1252 can define an interior volume 1204 that in some embodiments is hermetically sealed off from the area 1206 outside of the implantable housing 1252. The implantable monitor device 102 can include optical chemical sensor 206 which can include one or more chemical sensor elements. The optical chemical sensor 206 can be covered by a cover layer or a window 1202.

The window 1202 can be formed from a permeable material, such as an ion permeable polymeric matrix material. Many different materials can be used as the ion permeable polymeric matrix material. In some embodiments, the ion permeable polymeric matrix material can be a hydrogel. In some embodiments, the ion permeable polymeric material can be polyhydroxyethyl methacrylate (polyHEMA) either as a homopolymer or a copolymer including the same. The ion permeable polymeric matrix material(s) can be chosen based on its permeability to one or more of an electrolyte, a protein, a sugar, a hormone, a peptide, an amino acid, or a metabolic product. In some embodiments, the window 1202 can be opaque to the passage of light in one or more of the visible, ultraviolet (UV), or near-infrared (NIR) frequency spectrums.

The implantable monitor device 102 can further include circuitry 1208. The circuitry 1208 can include various components, such as components 1214, 1216, 1218, 1220, 1222, and 1224. In some embodiments, these components can be integrated and in other embodiments these components can be separate. In some embodiments, the components can include one or more of control circuitry (microprocessor, microcontroller, an ASIC, or the like), memory circuitry (such as random access memory (RAM) and/or read only memory (ROM)), recorder circuitry, telemetry circuitry, chemical sensor interface circuitry, one or more temperature sensors, temperature sensor control circuitry, power supply circuitry (which can include one or more batteries), normalization circuitry, chemical sensor control circuitry, and the like. In some embodiments, recorder circuitry can record the data produced by the chemical sensor and record time stamps regarding the same. In some embodiments, the circuitry can be hardwired to execute various functions, while in other embodiments the circuitry can be implemented as instructions executing on a microprocessor or other computation device.

A telemetry interface 1226 can be provided for communicating with external devices such as a programmer, a home-based unit, and/or a mobile unit (e.g., a cellular phone, portable computer, etc.). In some embodiments, the telemetry interface 1226 can be provided for communicating with implanted devices such as a therapy delivery device (e.g. a pacemaker, cardiovertor-defibrillator) or monitoring only device (e.g. an implantable loop recorder). In some embodiments, the circuitry can be implemented remotely, via either near-field, far-field, conducted, intra-body or extracorporeal communication, from instructions executing on any of the external or the implanted devices, etc. In some embodiments, the telemetry interface 1226 can be located within the implantable housing 1252. In some embodiments, the telemetry interface 1226 can be located in header 104.

An optical excitation assembly 1212 as well as optical detection assemblies 1210, 1258 can be in electrical communication with the circuitry 1208 within the interior volume 1204. In some embodiments, the control circuitry 1208 is configured to selectively activate the optical excitation 1212 and optical detection assemblies 1210, 1258. The optical excitation assembly 1212 can be configured to cast light onto one or more chemical sensing elements of the optical chemical sensor 206. The optical detection assemblies 1210, 1258 are configured to receive light from the one or more chemical sensor elements.

While FIG. 12 shows light rays reflecting and/or absorbing near the bottom of the well/sensing elements, it will be appreciated that this is merely for ease of illustration and that the entire sensing element can be exposed to light from the optical excitation assemblies and therefore contribute to generating a response that can be detected by the optical detection assemblies.

Referring now to FIG. 13, a schematic diagram is shown of components of an implantable monitor device 102 in accordance with various embodiments herein. It will be appreciated that some embodiments can include additional elements beyond those shown in FIG. 13. In addition, some embodiments may lack some elements shown in FIG. 13. The implantable monitor device 102 can gather information through one or more sensing channels. A microprocessor 1302 can communicate with a memory 1304 via a bidirectional data bus. The memory 1304 can include read only memory (ROM) or random access memory (RAM) for program storage and RAM for data storage, or any combination thereof. The implantable monitor device 102 can also include one or more chemical sensors 108 and one or more chemical sensor channel interfaces 1306 which can communicate with a port of the microprocessor 1302. The chemical sensor channel interface 1306 can include various components such as analog-to-digital converters for digitizing signal inputs, sensing amplifiers, registers which can be written to by the control circuitry in order to adjust the gain and threshold values for the sensing amplifiers, source drivers, modulators, demodulators, multiplexers, and the like. A telemetry interface 1326 is also provided for communicating with external devices such as a programmer, a home-based unit, and/or a mobile unit (e.g., a cellular phone, portable computer, etc.), implanted devices such as a pacemaker, cardiovertor-defibrillator, loop recorder, and the like.

### Temperature

In various embodiments, a temperature reference can be used for the calibration process. The temperature reference can be provided by internal temperature sensors of the device, by including a temperature sensor on or in a package, by including a passive sensor on or in a package like a liquid crystal temperature indicator (such as for “within range to permit valid test” indicators), or a combination of these or other temperature sensors or temperature inputs (such as an input from a user).

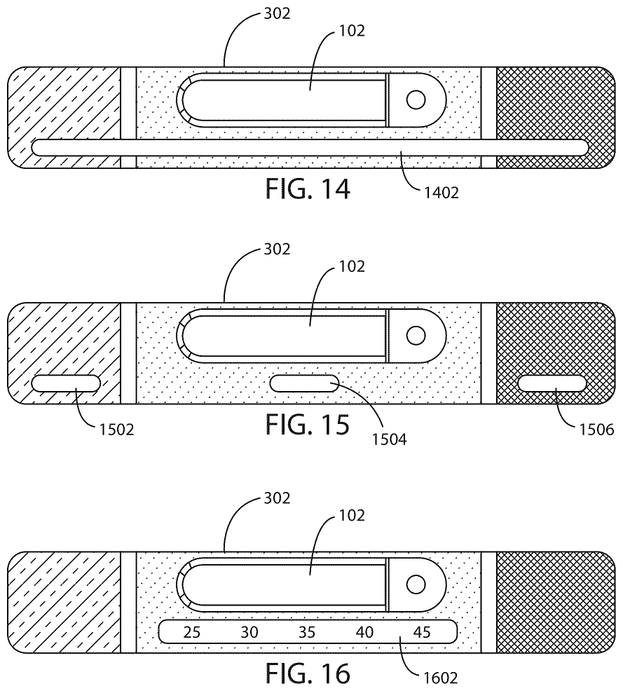

Referring now to FIG. 14, a schematic view is shown of an implantable sensor system including an implantable monitor device within a package in accordance with various embodiments herein. As before, implantable monitor device 102 is disposed within package 302. In this example, one temperature sensing strip 1402 covers at least part of all chambers of the package 302. The temperature sensing strip 1402 can be formed with a thermochromic material (such as a dye) or a liquid crystal so that if the temperature is within a desired range the temperature sensing strip 1402 is a particular color. In use, in one approach, all of the strip 1402 must be within an acceptable color band (indicating that all chambers are an appropriate temperature) for calibration and/or testing operations to commence.

Referring now to FIG. 15, a schematic view is shown of an implantable sensor system including an implantable monitor device within a package in accordance with various embodiments herein. As before, implantable monitor device 102 is disposed within package 302. Temperature sensors 1502, 1504, and 1506 can be disposed in or over chambers of the package 302. In some embodiments, the temperature sensors 1502, 1504, and 1506, can be a thermocouple, thermistor, resistance based temperature sensor, semiconductor-based temperature sensor or the like. However, in some embodiments, the temperature sensors can be passive sensors, such as sensors made with thermochromic materials or liquid crystal materials. Regardless, the user can evaluate temperature and proceed if it is within a desirable range. In some embodiments, passive sensors can produce a color change that is variable across a range of temperatures. In other embodiments, passive sensors can be configured to show one color if the temperature is acceptable and a different color if the temperature is unacceptable (e.g., “go” or “no go”). In some embodiments, the user or a component of the system can input a temperature value into the implantable monitor device 102. In some embodiments, the implantable monitor 102 can receive a signal regarding temperature directly from the temperature sensor(s).

Referring now to FIG. 16, a schematic view of an implantable sensor system including an implantable monitor device within a package in accordance with various embodiments herein. As before, implantable monitor device 102 is disposed within package 302. In this embodiment, the temperature sensor 1602 can be configured so as to provide temperature values based on indicia in combination with thermochromic materials.

Temperatures information can be used in various ways herein. In some embodiments, the system can use the temperature data (internal or external) to allow the calibration to proceed or not. In some embodiments, the system can adjust temporal boundaries of the step response (see, e.g., FIGS. 8-10) based on the prevailing temperature (as the sensor response speed can be affected by temperature).

### Methods

Many different methods are contemplated herein, including, but not limited to, methods of making, methods of using, and the like. Aspects of system/device operation described elsewhere herein can be performed as operations of one or more methods in accordance with various embodiments herein.

In various embodiments, operations described herein and method steps can be performed as part of a computer-implemented method executed by one or more processors of one or more computing devices. In various embodiments, operations described herein and method steps can be implemented instructions stored on a non-transitory, computer-readable medium that, when executed by one or more processors, cause a system to execute the operations and/or steps.

In an embodiment, a method of calibrating an implantable chemical sensor is included. The method can include breaking a first seal of the package to provide fluid communication between the first chamber and a second chamber of the package. In some embodiments, the method can also include prompting a system user to break the first seal. The method can also include measuring a concentration of the solute in the first chamber of the package. The method can also include comparing the measured concentration in the first chamber against a known concentration of the solute.

In an embodiment, the method can further include measuring a concentration of a solute in a first chamber of a package prior to the breaking a first seal of the package to provide fluid communication between the first chamber and a second chamber of the package.

In an embodiment, the method can further include determining a calibration correction based on a comparison of the measured concentration in the first chamber against a known concentration of the solute.

In an embodiment, the method can further include waiting for the implantable chemical sensor to reach an equilibrium value after the operation of breaking the first seal of the package and determining a response time of the implantable chemical sensor based on an elapsed time.

In an embodiment, the method can further include breaking a second seal of the package to provide fluid communication between the first chamber and a third chamber of the package and measuring a concentration of the solute in the first chamber of the package. In some embodiments, the method can also include prompting a system user to break the second seal.

In an embodiment, the method can further include waiting for the implantable chemical sensor to reach an equilibrium value after the operation of breaking the second seal of the package and determining a response time of the implantable chemical sensor based on an elapsed time.

In an embodiment of the method, the first chamber is filled with a liquid prior to the breaking a first seal of the package to provide fluid communication between the first chamber and a second chamber of the package. In an embodiment of the method, the first chamber is not filled with a liquid prior to the breaking a first seal of the package to provide fluid communication between the first chamber and a second chamber of the package.

In some embodiments, a method herein can also include prompting a system user that the device is ready for implant after the calibration operations are complete.

In an embodiment, the method can further include measuring temperature and adjusting a response time and/or a response time comparison value based on the same.

In an embodiment, the method can further include measuring and/or receiving a temperature value and terminating calibration if the temperature value is not within a predetermined range.

It should be noted that, as used in this specification and the appended claims, the singular forms “a,” “an,” and “the” include plural referents unless the content clearly dictates otherwise. Thus, for example, reference to a composition containing “a compound” includes a mixture of two or more compounds. It should also be noted that the term “or” is generally employed in its sense including “and/or” unless the content clearly dictates otherwise.

It should also be noted that, as used in this specification and the appended claims, the phrase “configured” describes a system, apparatus, or other structure that is constructed or configured to perform a particular task or adopt a particular configuration. The phrase “configured” can be used interchangeably with other similar phrases such as arranged and configured, constructed and arranged, constructed, manufactured and arranged, and the like.

All publications and patent applications in this specification are indicative of the level of ordinary skill in the art to which this invention pertains. All publications and patent applications are herein incorporated by reference to the same extent as if each individual publication or patent application was specifically and individually indicated by reference.

As used herein, the recitation of numerical ranges by endpoints shall include all numbers subsumed within that range (e.g., 2 to 8 includes 2.1, 2.8, 5.3, 7, etc.).

The headings used herein are provided for consistency with suggestions under 37 CFR 1.77 or otherwise to provide organizational cues. These headings shall not be viewed to limit or characterize the invention(s) set out in any claims that may issue from this disclosure. As an example, although the headings refer to a “Field,” such claims should not be limited by the language chosen under this heading to describe the so-called technical field. Further, a description of a technology in the “Background” is not an admission that technology is prior art to any invention(s) in this disclosure. Neither is the “Summary” to be considered as a characterization of the invention(s) set forth in issued claims.

The embodiments described herein are not intended to be exhaustive or to limit the invention to the precise forms disclosed in the following detailed description. Rather, the embodiments are chosen and described so that others skilled in the art can appreciate and understand the principles and practices. As such, aspects have been described with reference to various specific and preferred embodiments and techniques. However, it should be understood that many variations and modifications may be made while remaining within the spirit and scope herein.

1. A chemical sensor system comprising:
a package; and
an implantable monitor device, the implantable monitor device comprising an optical chemical sensor;
wherein the implantable monitor device is disposed within the package;
a first aqueous solution, the first aqueous solution comprising a solute; and
wherein the first aqueous solution is disposed within the package.

2. The chemical sensor system of claim 1, the solute comprising an electrolyte.

3. The chemical sensor system of claim 1, wherein the first aqueous solution has a pH of 7.0 to 7.8.

4. The chemical sensor system of claim 1, the optical chemical sensor comprising a hydrogel layer.

5. The chemical sensor system of claim 1,
the package comprising a first chamber;
the package comprising a second chamber; and
the package comprising a seal;
wherein the first aqueous solution is disposed within the first chamber;
the chemical sensor system further comprising a second aqueous solution, wherein the second aqueous solution is disposed in the second chamber;
wherein the seal is separates the first chamber and the second chamber; and
wherein the seal is frangible.

6. The chemical sensor system of claim 1, further comprising a temperature sensor, wherein the temperature sensor is disposed on or in the package.

7. A chemical sensor system comprising:
a package, the package comprising
a first chamber;
a second chamber; and
a seal;
wherein the seal is disposed separates the first chamber and the second chamber;
wherein the seal is frangible;
an implantable monitor device, the implantable monitor device comprising an optical chemical sensor;
wherein the implantable monitor device is disposed within the first chamber;
a first aqueous solution, the first aqueous solution comprising a solute; and
wherein the first aqueous solution is disposed within the second chamber.

8. The chemical sensor system of claim 7, wherein the package is hermetically sealed.

9. The chemical sensor system of claim 7, wherein the first aqueous solution has a pH of 7.0 to 7.8.

10. The chemical sensor system of claim 7, the package further comprising:
a third chamber; and
a second seal, wherein the second seal is frangible; and
the chemical sensor system further comprising a second aqueous solution, wherein the second aqueous solution is disposed within the third chamber, the second aqueous solution comprising
a solute, wherein the solute of the second aqueous solution is at a different concentration than in the first aqueous solution.

11. The chemical sensor system of claim 7, further comprising a temperature sensor, wherein the temperature sensor is disposed on or in the package.

12. The chemical sensor system of claim 11, wherein the temperature sensor is disposed over or in at least part of each of the chambers.

13. A method of calibrating an implantable chemical sensor comprising:
breaking a first seal of the package to provide fluid communication between the first chamber and a second chamber of the package;
measuring a concentration of the solute in the first chamber of the package; and
comparing the measured concentration in the first chamber against a known concentration of the solute.

14. The method of calibrating an implantable chemical sensor of claim 13, further comprising measuring a concentration of a solute in a first chamber of a package prior to the breaking a first seal of the package to provide fluid communication between the first chamber and a second chamber of the package.

15. The method of calibrating an implantable chemical sensor of claim 13, further comprising determining a calibration correction based on the compared concentration.

16. The method of calibrating an implantable chemical sensor of claim 13, further comprising waiting for the implantable chemical sensor to reach an equilibrium value after the operation of breaking the first seal of the package and determining a response time of the implantable chemical sensor based on an elapsed time.

17. The method of calibrating an implantable chemical sensor of claim 13, further comprising:
breaking a second seal of the package to provide fluid communication between the first chamber and a third chamber of the package; and
measuring a concentration of the solute in the first chamber of the package.

18. The method of calibrating an implantable chemical sensor of claim 17, further comprising waiting for the implantable chemical sensor to reach an equilibrium value after the operation of breaking the second seal of the package and determining a response time of the implantable chemical sensor based on an elapsed time.

19. The method of calibrating an implantable chemical sensor of claim 13, further comprising measuring temperature and adjusting a response time and/or a response time comparison value based on the same.

20. The method of calibrating an implantable chemical sensor of claim 13, further comprising measuring and/or receiving a temperature value and terminating calibration if the temperature value is not within a predetermined range.
